(19) United States
(12) Patent Application Publication
(10) Pub. No.: US 2017/0067912 A1
(43) Pub. Date: Mar. 9, 2017

(54) COMPOSITIONS AND METHODS FOR DIAGNOSING BARRETT'S ESOPHAGUS STAGES

(71) Applicant: The Penn State Research Foundation, University Park, PA (US)

(72) Inventors: Douglas STAIRS, Breinigsville, PA (US); Julie MASSE, Saint-Ouen d'Aunis (FR)

(21) Appl. No.: 15/121,199

(22) PCT Filed: Mar. 13, 2015
(86) PCT No.: PCT/US15/20436

§ 371 Date: Aug. 24, 2016

Related U.S. Application Data
(60) Provisional application No. 61/952,568, filed on Mar. 13, 2014.

(51) Int. Cl. G01N 33/68; G01N 33/577

(52) U.S. Cl. CPC G01N 33/6893; G01N 33/577; G01N 2800/06; G01N 2800/56

(57) ABSTRACT

Provided is an immunohistochemistry panel that facilitates the discrimination between the early and late stages of Barret's esophagus in a method that involves testing a biological sample for expression of CDX2, p120ctn, c-Myc and Jagged1 proteins, comparing the amount of the CDX2, p120ctn, c-Myc and Jagged1 proteins to reference values, and providing a diagnosis of or aiding in a physician's diagnosis, of the individual as having high-grade dysplasia (HGD) or esophageal adenocarcinoma (EAC) by determining less CDX2 protein relative to non-dysplastic Barrett's esophagus (ND-BE) and low-grade dysplasia (LGD) CDX2 protein values, but more CDX2 protein than a normal CDX2 protein reference value; and less p120ctn protein relative to ND-BE, LGD and normal 120ctn protein reference values; and increased c-Myc protein relative to ND-BE and LGD protein reference values; and increased Jagged1 protein relative to normal and ND-BE Jagged1 protein reference values. Kits for making the protein determinations are also provided.

### CROSS-REFERENCE TO RELATED APPLICATIONS

This application claims priority to U.S. provisional patent application No. 61/952,568, filed Mar. 13, 2014, the disclosure of which is incorporated herein by reference.

### FIELD

The present disclosure relates generally to approaches for determining risk for and/or aiding in the prognosis and/or diagnosis of cancer, and in particular, esophageal adenocarcinoma.

### BACKGROUND

Barrett's esophagus (BE) is a known precursor lesion for esophageal adenocarcinoma (EAC) and its presence significantly increases the risk of progression to EAC (1). The progression through a metaplasia-dysplasia-adenocarcinoma sequence is slow and unpredictable. Currently, histologic assessment of BE biopsies remains the gold standard for diagnosis in these patients (2). Detection of non-dysplastic BE (ND-BE) and low-grade dysplasia (LGD) necessitates repeated surveillance endoscopy since these pose a relatively lower risk of progression while high-grade dysplasia (HGD) and EAC may warrant endoscopic ablative techniques and/or esophagectomy (3, 4). Therefore, the transition from patients that present earlier stages of the disease with low risk to develop cancer (ND-BE and LGD) to patients with late stages with higher risk to progress to cancer (HGD and EAC) is a crucial event in the disease and accurate staging is necessary for optimal patient care. Although critical, the histological examination remains subjective and is complicated by significant interobserver variability (5-7). Therefore, there is a need for new, accurate markers that can be used objectively as an adjunct to histologic assessment, thus improving upon the correct diagnosis and the proper management of patients.

### SUMMARY

The present disclosure provides compositions and method that are useful for predicting risk of developing EAC, and/or generating a prognosis for an individual at risk for developing EAC, and/or generating a treatment approach for an individual who is at risk for developing EAC, and for combining methods disclosed herein with EAC treatments. In arriving at the present disclosure, to accurately distinguish low risk and high risk patients, we analyzed the expression of c-Myc, CDX2, and Jagged1 as well as p120ctn expression. In particular, in the present disclosure we show, through the use of an immunohistochemistry panel, the discrimination between the early and late stages of Barrett's Esophagus. We focused on the expression of CDX2, p120-catenin (p120-ctn), c-Myc, and Jagged1 which as described further herein are differentially regulated during the progression from ND-BE to EAC. In order to validate the diagnostic utility of the present approach, CDX2, p120ctn, c-Myc and Jagged1 expression were assessed by immunohistochemistry (IHC) and semi-quantitative scoring on 101 BE biopsies. Scores were integrated using principal component analysis (PCA) and receiver operating characteristic (ROC) curve. We confirmed that expression of the four proteins is significantly altered between ND-BE/LGD and HGD/EAC stages of BE. PCA demonstrated the ability of this panel of protein to segregate early and late stages. The ROC curve showed that this panel has a potential to aid in the diagnosis of Barrett's esophagus with both high specificity and sensitivity. Using the four proteins as a panel can improve the discrimination of the early and late stages of Barrett's esophagus and accordingly can aid in the diagnosis and management of patients. Those skilled in the art will recognize that, while demonstrated in the instant disclosure using IHC, any other approach that can be used to ascertain the relative amounts and/or cellular localization of the proteins of interest can be used.

In view of the foregoing, it will be apparent that in general, the instant disclosure comprises a method for staging an esophageal condition in an individual at risk for or suspected of having the esophageal condition. The approach is particularly useful for determining whether an individual has HGD or EAC, and thus the individual will require more aggressive or different treatment than an individual with BE or LGD. As such, the method can result in the diagnosis, and/or can aid in a physician's diagnosis of HGD or EAC, and can distinguish these advanced forms of an esophageal condition from BE or LGD, and in certain embodiments encompasses making a treatment recommendation, and/or treating the individual, such as with a surgical intervention.

The method generally comprises testing a biological sample from the individual for expression of CDX2, p120ctn, c-Myc and Jagged1 protein, and comparing the amount of the CDX2, p120ctn, c-Myc and Jagged1 proteins to reference values. The diagnosis of HGD and/or EAC is indicated by determining:
i) less CDX2 protein relative to non-dysplastic ND-BE and LGD CDX2 protein values, but more CDX2 protein than a normal CDX2 protein value; and
ii) less p120ctn protein relative to ND-BE, LGD and normal 120ctn protein reference values; and
iii) increased c-Myc protein relative to ND-BE and LGD protein reference values; and
iv) increased Jagged1 protein relative to normal and ND-BE Jagged1 reference values.

### DESCRIPTION OF THE FIGURES

FIG. 1: Photomicrographs depicting neoplasia in Barrett's esophagus. A, Normal esophageal squamous mucosa (H&E×100). B, Non-dysplastic Barrett's mucosa (H&E×100). C, Low-grade dysplasia (H&E×100). D, High-grade dysplasia (H&E×100). E, Intramucosal adenocarcinoma (H&E×100). Scale bars=20 μm. (H&E represents Hematoxylin and Eosin)

FIG. 2: Immunohistochemical expression of nuclear CDX2 in Barrett's esophagus. A, Negative staining of CDX2 in normal esophageal squamous mucosa (CDX2 IHC×200). B&C, Non-dysplastic Barrett's mucosa and low-grade dysplasia showing strong nuclear CDX2 expression, respectively (CDX2 IHC×200). D&E, High-grade dysplasia and adenocarcinoma showing a down-regulation of nuclear CDX2 expression, respectively (CDX2 IHC×200). F, Bar graph showing that the nuclear CDX2 down-regulation in high risk disease is statistically significant (p≦0.001). Scale bars=50 μm.

FIG. 3: Immunohistochemical expression of membranous p120ctn in Barrett's esophagus. A, Strong, membranous staining of p120ctn in normal esophageal squamous mucosa (p120ctn IHC×200). B, Non-dysplastic Barrett's mucosa showing intense staining of the cell membranes (p120ctn IHC×200). C, Low-grade dysplasia showing moderate membranous and cytoplasmic expression of p120ctn, (p120ctn IHC×200). D&E, High-grade dysplasia and adenocarcinoma showing a down-regulation of membranous p120ctn expression and a relocalization to the cytoplasm, (p120ctn IHC×200). F, Bar graph showing that the membranous staining of p120ctn is significantly down-regulated in high risk disease (p≦0.001). Scale bars=50 μm.

FIG. 4: Immunohistochemical expression of nuclear c-Myc in Barrett's esophagus. A, No expression of c-Myc was detected in normal esophageal squamous mucosa (c-Myc IHC×200). B&C, Non-dysplastic Barrett's mucosa and low grade dysplasia showing weak and scattered staining of nuclei, respectively (c-Myc IHC×200). D&E, High grade dysplasia and adenocarcinoma showing a strong nuclear expression of c-Myc, respectively (c-Myc IHC×200). F, Bar graph showing that the nuclear staining is significantly up-regulated in high risk disease (p≦0.001). Scale bars=50 μm.

FIG. 5: Immunohistochemical expression of membranous Jagged1 in Barrett's esophagus. A Weak staining of Jagged1 in normal esophageal squamous mucosa (Jagged1 IHC×200). B, Non-dysplastic Barrett's mucosa showing weak membranous staining of Jagged1 (Jagged1 IHC×200). C, Low-grade dysplasia showing a weak membranous and cytoplasmic expression of Jagged1, (Jagged1 IHC×200). D&E, High-grade dysplasia and adenocarcinoma showing moderate membranous and cytoplasmic expression and a relocalization to the cytoplasm, respectively (Jagged1 IHC×200). F, Bar graph showing that the membranous Jagged1 staining is significantly increased in high risk disease (p≦0.001). Scale bars=50 μm.

FIG. 6: Statistical analysis of the immunohistochemical staining. A, Principal component analysis of the immunohistochemistry staining scores was generated from low risk patients (clear circles) and high risk patients (dark circles). A demarcation line was added for easy visualization. B, The box plot represents the calculated distribution over the low and high risk samples and showed a significant difference for each group (p≦0.001). C, The receiving operating curve (ROC) depicts the accuracy of CDX2, p120ctn, c-Myc and Jagged1 expression scores as markers of diagnosis with a confidence interval of 95%. The area under curve for this ROC was 0.956.

### DETAILED DESCRIPTION

The present disclosure provides compositions and method that are useful for predicting risk of developing HGD and/or EAC, and/or generating a prognosis for an individual at risk for developing HGD and/or EAC, and/or generating a treatment approach for an individual who is at risk for developing HGD and/or EAC, and for combining methods disclosed herein with HGD and/or EAC treatments. In developing the invention, to accurately distinguish low risk and high risk patients, we analyzed the expression of c-Myc, CDX2, and Jagged1 as well as p120ctn expression.

The expression of the four proteins (c-Myc, CDX2, p120ctn and JAG-1) was examined retrospectively with immunohistochemistry and semi-quantitative scoring to assess their expression in BE, LGD, HGD and EAC. Following scoring, we analyzed each marker for differential expression between low risk (BE and LGD) and high risk (HGD and EAC) patients. Thus, in embodiments, the method comprises testing for any one or any combination of c-Myc, CDX2, p120ctn and JAG-1. In embodiments, the testing comprises determining all four markers. In an embodiment, no other markers are tested, or only markers for disorders other than EAC are tested in addition to the four EAC markers and combinations thereof that are described here.

In general, the method comprising testing for such markers in a biological sample that is obtained from an individual. Any biological sample that comprises or would be expected to comprise the markers if the individual was at risk for developing HGD and/or EAC, or the individual has HGD and/or EAC and is undergoing therapy for it, can be used. The biological sample can be tested directly or it can be subjected to a processing step before testing. The biological sample can be a liquid biological sample, or it can be a sample of tissue, such as a sample of esophageal tissue. In embodiments, the method is performed using a biopsy or other tissue obtained from the individual. In embodiments, the method comprises an immunodetection approach, which typically involves forming a complex between one or more markers from the biological sample and a binding partner, such as an antibody. In embodiments, one or more binding partners comprising one or more detectable labels can be used. By measuring a signal from the detectable label, the presence or absence or the amount of any one or any combination of the markers can be determined Thus, the disclosure includes immunohistochemical based approaches that are used for quantifying the amounts of the markers in the sample, and can further be used to determine the sub-cellular localization of the proteins, which as further described below is also informative.

The amount of any one of the markers or any combination thereof can be compared to a suitable reference. The reference to which the markers can be compared include but are not limited to samples obtained from individuals who do not have the particular condition for which a diagnosis/or prognosis is sought, or who have a known stage of the condition. Thus, a reference that can be used in the approaches of this disclosure includes use of a normal reference. A “normal” reference is a value obtained from measuring the expression and/or sub-cellular localization in one or more individuals who do not have BE, LGD, HGD, or EAC. Any reference value used in the method of this disclosure, including but not limited to the normal reference value, can include or be derived from matched controls (i.e., matched for age, sex, or other demographics), a standardized curve(s), and/or experimentally designed controls such as known input marker, such as an known protein amount, used to normalize experimental data for qualitative or quantitative determination of the markers for mass, molarity, concentration and the like. The reference level may also be depicted graphically, such as an area on a graph or an area under a curve, or can be provided as a numerical value, or an absolute value, and/or an intensity value, such as an intensity of signal from an IHC assay. In certain embodiments the reference comprises standardized value(s) based on previous analysis of the markers from one or more individuals who have BE, LGD, HGD, or EAC. Combinations of such references are also used. In embodiments the disclosure includes, if desired, determining relative cellular locations for the markers, such as differences in nuclear localization, or translocation from the cytoplasm or vice versa, or membrane locations of the proteins, as between experimental and reference samples. For example, we determined that CDX2 localized to the nucleus is frequently present in ND-BE and LGD samples, but in HGD and EAC samples nuclear CDX2 was decreased in intensity and was less frequently detected. Because there is typically no detectable CDX2 in normal cells, merely detecting CDX2 is indicative of a non-normal sample in respect of any of ND-BE, LGD, HGD and EAC. For p120ctn, it is present at the plasma membrane in normal esophageal squamous mucosa with strong intensity in IHC, and has a strong and well defined expression pattern at the membrane of the columnar epithelial cells of ND-BE samples, whereas LGD samples have p120ctn at the membrane but, it is also mislocalized in the cytoplasm. While p120ctn expression was also partially mislocalized to the cytoplasm in HGD and EAC, it was significantly decreased in HGD and EAC samples. While there is no detectable immunoreactivity for c-Myc in normal esophageal squamous mucosa, it is weakly detectable in the nucleus of columnar cells in ND-BE and LGD samples, but it is much more strongly expressed in the nucleus of HGD and EAC samples. In HGD and EAC samples, Jagged1 is highly expressed relative to ND-BE, is present in a large proportion of cells and is highly diffuse in both the membrane and cytoplasm. Thus, the present disclosure also includes, if desired, analyzing the sub-cellular localization of one or any of the four proteins described above as an adjunct to determining their relative amounts. Further, the disclosure also includes if desired determining the number of cells in a sample that express the protein(s), and where such proteins are most frequently localized.

In certain embodiments, the disclosure includes sequential testing of the markers over a period of time, such as a treatment period to monitor the progress of a therapeutic approach. In embodiments, the method involves testing for one or more of the markers and recommending to a physician and/or performing a medical intervention, such as endoscopic ablative techniques or resection due to, for example, a risk of progression to HGD or EAC. In certain embodiments, determining an amount of one or more of the markers in an amount above or below a reference is a diagnosis of HGD or EAC, or aids in a physician's diagnosis of HGD/EAC, or aids in providing a prognosis for the individual from which the sample was obtained. In embodiments, the disclosure includes determining differential expression of markers as further described herein between early stages and late stages of the disease. For example, we have discovered that CDX2 and p120ctn are significantly decreased in HGD and EAC samples compared with BE and LGD samples, respectively, while c-Myc expression showed a significant increase in HGD/EAC patient samples compared with BE/EAC. Further, Jagged1 expression is also increased significantly between BE/LGD and HDG/EAC samples. Thus, in one embodiment, the method comprises testing a biological sample obtained or derived from an individual for expression of CDX2, p120ctn, c-Myc and Jagged1 protein, and comparing the amount of the CDX2, p120ctn, c-Myc and Jagged1 proteins to reference values. Based on the test results, HGD and/or EAC is diagnosed, or aids in a physician's diagnosis, by determining:
i) less CDX2 protein relative to non-dysplastic ND-BE and LGD CDX2 protein values, but more CDX2 protein than a normal CDX2 protein value; and
ii) less p120ctn protein relative to ND-BE, LGD and normal 120ctn protein reference values; and
iii) increased c-Myc protein relative to ND-BE and LGD protein reference values; and
iv) increased Jagged1 protein relative to normal and ND-BE Jagged1 reference values.

In an embodiment, the method further comprises determining i), ii), iii), and iv), and recommending and/or performing a surgical intervention. In one embodiment, the surgical intervention comprises performing an endoscopic ablative technique or an esophagectomy. In an embodiment, the ablative technique comprises use of thermal energy to reduce or eliminate cancer cells, such as by radiofrequency, laser, microwave, ultrasound, or cryoablation. In other embodiments, the ablative technique comprises chemical (ethanol or acetic acid) ablation.

In certain approaches the disclosure includes fixing the determination of the markers in a tangible medium of expression, such as a digitized file, compact disk, a paper-based report, and the like. In embodiments, the determination comprises a value that designates a immunohistochemical staining intensity, and/or an immunoreactivity score (IRS). The disclosure includes communicating the result to a health care or insurance provider.

Kits for testing as described herein are also included. The kits can include primers or other nucleic-acid based probes, or reagents for immunohistochemistry. In embodiments, the kit provides reagents, such as specific binding partners, for use in immunohistochemistry based testing for any one or combination, or all of c-Myc, CDX2, p120ctn and JAG-1. In embodiments, at least one of the binding partners is a novel binding partner. In embodiments, the kits comprise primary antibodies directed to c-Myc, CDX2, p120ctn and JAG-1, such as for use in an IHC assay, wherein detectably labeled secondary antibodies are used to detect complexes of primary antibodies and the proteins. In embodiments, the antibodies directed to c-Myc, CDX2, p120ctn and JAG-1 are the only monoclonal antibodies provided with the kit. In embodiments, the secondary detection antibodies, which may be monoclonal or polyclonal, are labeled with an enzyme that produces a detectable signal when exposed to a substrate that produces a detectable signal when contacted by the enzyme. In alternative embodiments the kit comprises four detectably labeled monoclonal antibodies that separately bind with specificity to CDX2, p120ctn, c-Myc and Jagged1. In certain embodiments, the disclosure includes an article of manufacture comprising one or more such reagents, suitable containers, and packaging, wherein the packaging contains printed material which provides an indication that the contents of the package are to be used for diagnosis, for aiding in the diagnosis, for staging, or for testing a sample for one or more markers that are indicative of BE, LGD, or HGD, or EAC. In an embodiment the printed material provides an indication that the article of manufacture is for diagnosing, or aiding in the diagnosis or staging a sample obtained from an individual who is at risk for developing or who has EAC.

The following Examples are intended to illustrate but not limit the disclosure.

### EXAMPLE 1

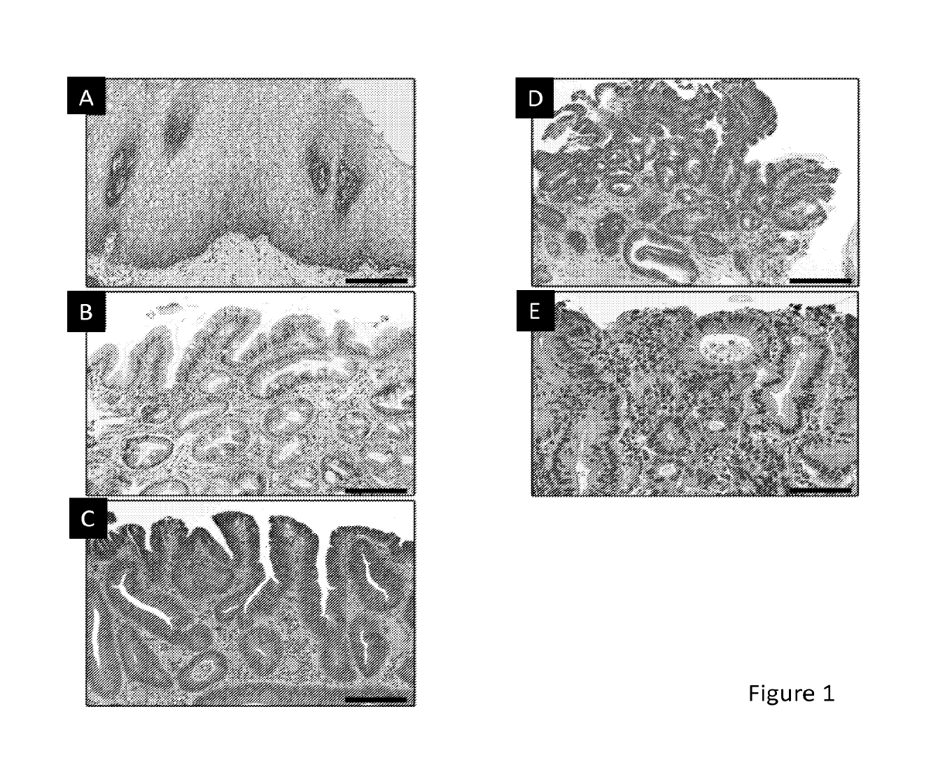

We identified 101 patient samples for a retrospective study with agreement on the final diagnosis between the original pathologist at the time of the diagnosis and the expert GI pathologist who reviewed the slides to generate data presented in the Examples of this disclosure. The age of patients ranged from 31 to 89 years (mean 64.4, median 65) with a male-to-female ratio of 4:1 (79 men and 20 women). All patients had endoscopically identifiable columnar mucosa and their biopsies were histologically classified as ND-BE (37 samples), LGD (12 samples), HGD (25 samples) and EAC (27 samples) (FIG. 1). These samples were subsequently used to perform IHC for CDX2, p120ctn, c-Myc and Jagged1 proteins.

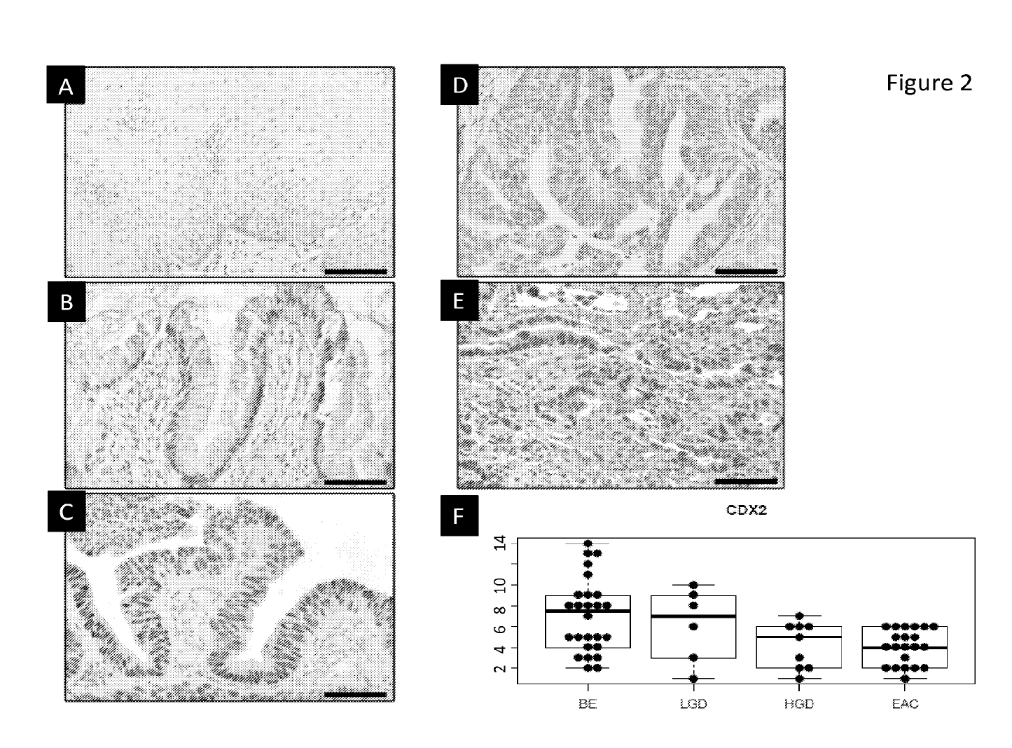

Immunohistochemical analysis of CDX2 showed no expression in normal esophageal squamous mucosa (FIG. 2A). Nuclear CDX2 staining was present in a large majority of cells in ND-BE and LGD samples (FIGS. 2B and 2C). In HGD and EAC samples, nuclear CDX2 staining was decreased in intensity as well as the percentage of cells stained (FIGS. 2D and 2E). Scoring analysis revealed a significant down-regulation of CDX2 expression between the early stages samples (ND-BE and LGD) and the later stages samples (HGD and EAC) (p≦0.004) (FIG. 2F).

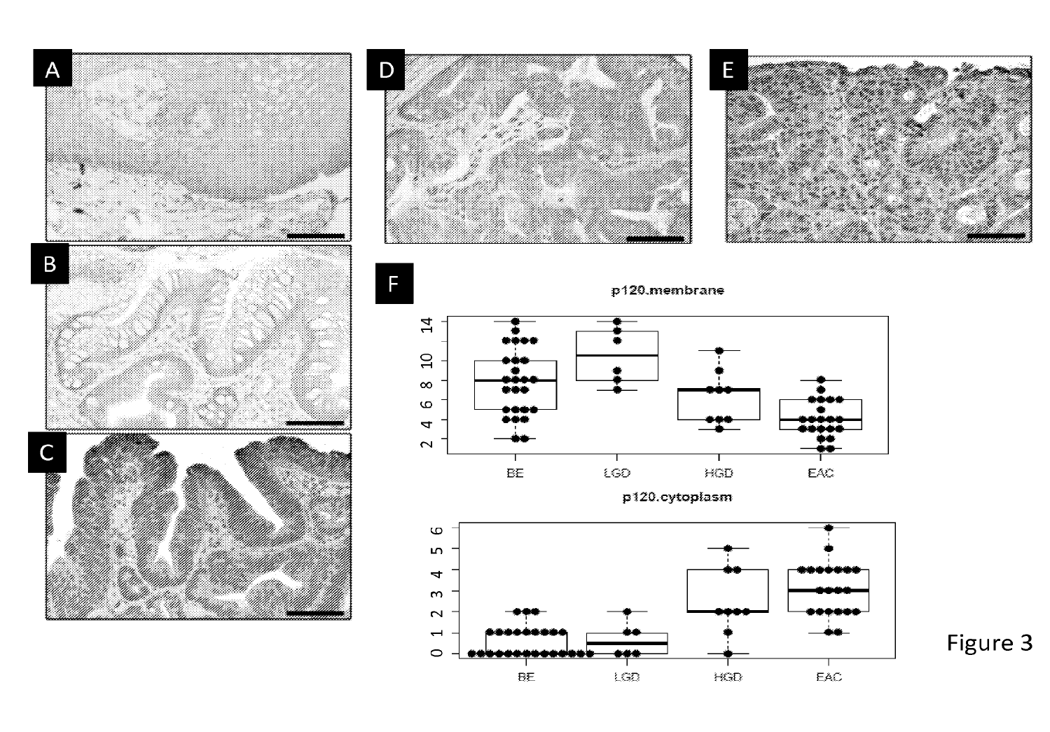

p120ctn expression was detected at the plasma membrane in normal esophageal squamous mucosa with strong intensity (FIG. 3A). p120ctn had a strong and well defined expression pattern at the membrane of the columnar epithelial cells of ND-BE samples (FIG. 3C). LGD samples showed an expression of p120ctn at the membrane as well as mislocalized in the cytoplasm of the cells (FIG. 3D). p120ctn expression was significantly decreased in HGD and EAC samples and the protein was partially mislocalized to the cytoplasm of the cells (FIGS. 3D and 3E). The expression of p120ctn was quantified and revealed a significant decrease in the later stages samples compared with earlier stages samples (p≦0.001), (FIG. 3F).

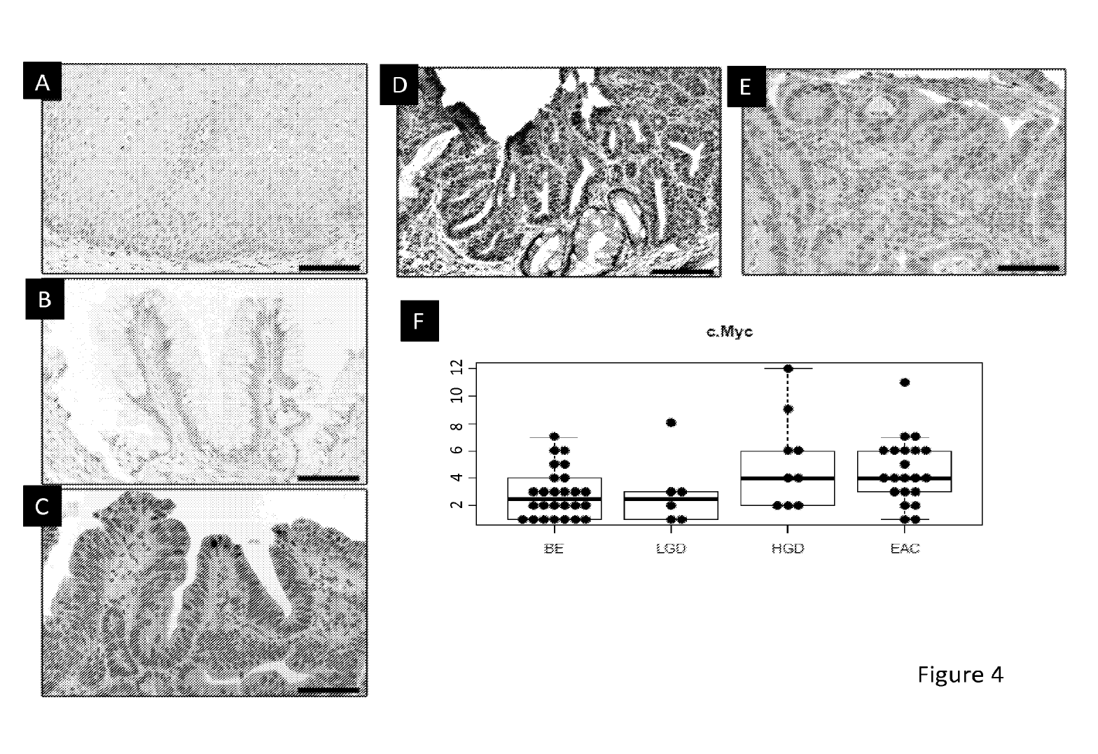

No immunoreactivity for c-Myc was observed in the normal esophageal squamous mucosa (FIG. 4A) Immunohistochemical staining for c-Myc showed a weak nuclear expression in the columnar cells in ND-BE and LGD samples (FIGS. 4B and 4C). In contrast, HGD and EAC samples had significantly stronger nuclear c-Myc expression (FIGS. 4D and 4E), suggesting that nuclear c-Myc expression increases during the progression of the disease (p≦0.02), (FIG. 4F).

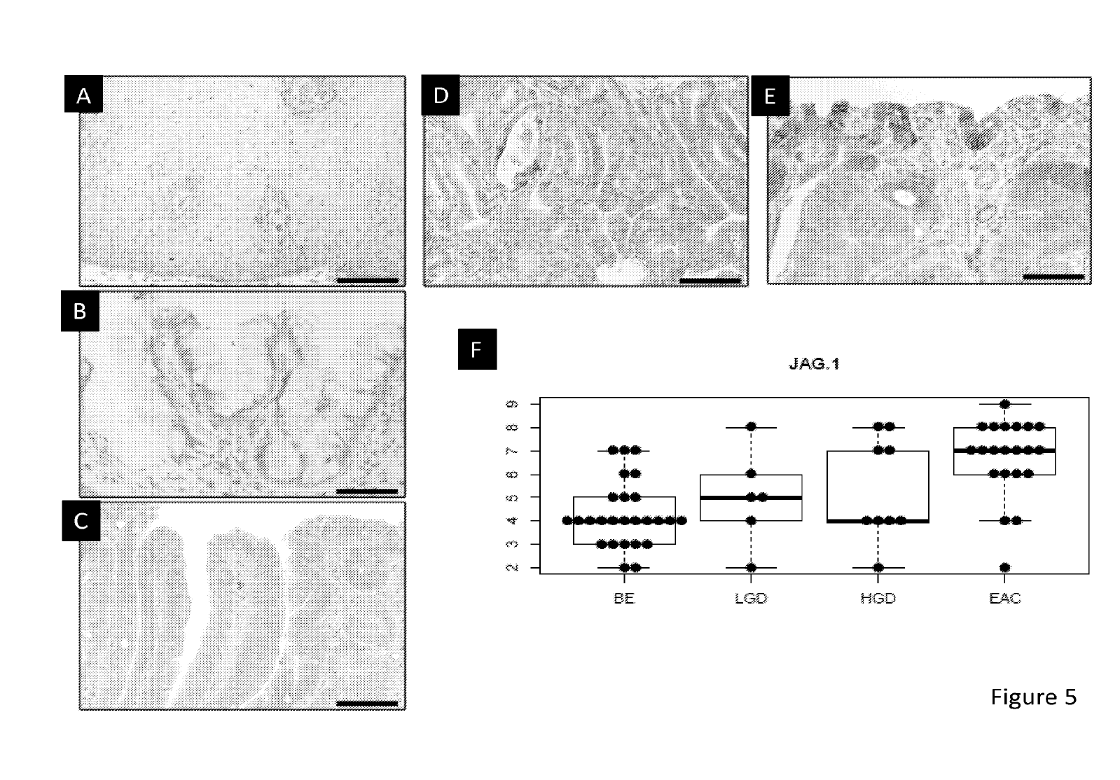

Immunohistochemical expression for Jagged1 protein in normal esophageal squamous mucosa was membranous but weak (FIG. 5A). Jagged1 was identified at the membrane of columnar cells of ND-BE samples (FIG. 5B). The same localization was observed in LGD samples but with additional diffuse staining in the cytoplasm of the cells (FIG. 5C). In HGD and EAC samples, Jagged1 was detected in a large proportion of cells and appeared to be highly diffuse; with moderate expression seen in both the membrane and cytoplasm (FIGS. 5D and 5E), respectively. Scoring of membranous Jagged1 expression showed a significant increase from earlier to later stages patient samples (FIG. 5F). Also, Jagged1 was significantly higher in HGD compared with LGD, illustrating the switch of expression occurring between these two stages (data not shown, p=0.026).

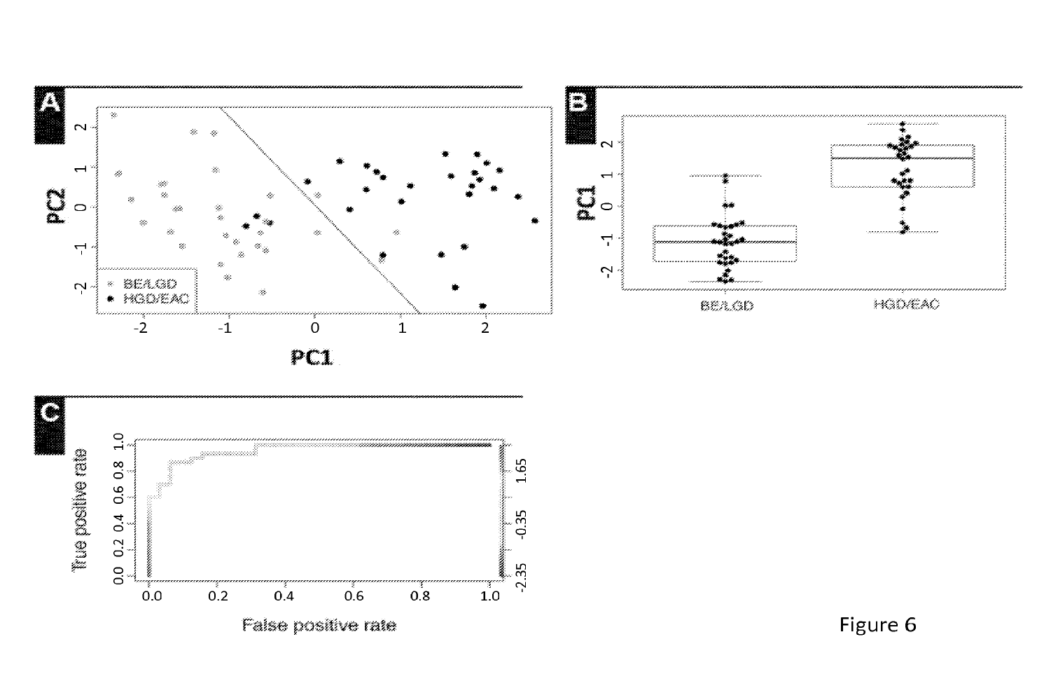

To determine the ability of these four markers to classify the stages of neoplasia (early stages=ND-BE and LGD and late stages=HGD and EAC), we utilized the scores obtained from the quantification of each of the four proteins together. An unsupervised principal component analysis was able to segregate samples into two groups—earlier stages patients and later stages patients (FIG. 6A). This observation was confirmed by the box plot graph of the first principal component that showed a significant change in the distribution of the samples over the two groups (p≦0.001) (FIG. 6B). Additionally, a ROC curve was plotted to determine the ability of the integrated IHC scores for the four proteins to distinguish ND-BE and LGD from HGD and EAC. ROC analysis provided 87.5% sensitivity and 86.7% specificity and area under the curve of 0.956 (FIG. 6C). Globally, the expression level of the four markers CDX2, p120ctn, c-Myc and Jagged1 can help to determine the stage of the disease and therefore the risk of progression of the BE neoplasia and the proper patient care.

It will be recognized from the foregoing that the methods of the instant disclosure are important because, for example, EAC is the predominant form of esophageal cancer in the United States (16) and EAC tumor incidence has experienced the highest rate of increase among all solid tumors during the past 30 years (17, 19). EAC is a highly aggressive disease and the late stage diagnosis of this cancer explains its poor 5-year survival rate of less than 20%. BE is considered as a risk factor for EAC, and prior to the present disclosure, the best predictive factor for the future development of a carcinoma in a given patient is the identification of BE with high grade dysplasia. Indeed, HGD has a poor prognosis due to the high rate of progression from HGD to EAC (19% per year) (13). Patients with ND-BE or LGD undergo frequent endoscopic surveillance for possible progression, while the presence of HGD leads to specific therapeutic actions including endoscopic ablative techniques or esophagectomy (3, 4). Therefore, the accurate diagnosis of LGD vs HGD is of paramount importance for the proper treatment of patients. However, the interpretation of histologic features of LGD and HGD is subjective (5-7). In particular, the discrimination between the upper spectrum of the LGD and the lower spectrum of HGD can be difficult, especially since the “moderate dysplasia” stage was eliminated. Prior to the present disclosure, and despite the effort to identify new diagnostic markers that could accurately stage late stages patients, no reliable markers have been previously available to be used in clinical practice and the diagnosis of BE, which was still based on histopathologic examination of the biopsies. Therefore, there is a great need for the diagnostic tools of the present disclosure, which will reduce subjectivity by increasing the accuracy of the histologic interpretation and optimize patient care, especially in challenging cases.

In this regard, and as will be appreciated by those skilled in the art from the foregoing data, we analyzed the expression of CDX2, p120ctn, c-Myc and Jagged1 and assessed the value of the combination of these four proteins to accurately stage BE patient's biopsy samples. By selecting proteins that are in different signaling pathways during the progression of Barrett's esophagus to EAC, the objective was to increase the diagnostic accuracy of BE and distinguish between early and late stages of the disease. Proper discrimination between the ND-BE/LGD and HGD/EAC will determine appropriate patient care, either enrollment in surveillance routine or the need for a more invasive treatment. In our cohort, nuclear CDX2 and p120ctn expression were significantly decreased between early and late stage patients whereas c-Myc and Jagged1 expression were significantly upregulated in the HGD/EAC groups. PCA analysis that integrates the score of each of these four proteins illustrates that a combinatorial protein expression analysis approach can accurately determine the stage of the BE neoplasia. These four proteins together (but not individually) comprise a novel panel that is expected to more accurately classify the samples between the early or late stages of the disease than does histologic analysis alone. Additionally, ROC curve analysis highlighted that CDX2, p120ctn, c-Myc and Jagged-1 are good indicators for stage segregation due to its high specificity and sensitivity.

Few other factors have also been evaluated for their potential as BE diagnosis markers. The area under the curve generated by ROC analysis ranged between 0.607 and 0.852 in prior studies (20-22). Using the present combination of markers, the area under the ROC curve is 0.956, indicating a more accurate diagnosis of the disease than previous studies and suggesting that the markers presented here could improve the classification of patients into earlier and later stage groups. Recently, AMACR (α-methylacyl coenzyme A racemase) has been shown to be useful for differentiating ND-BE, LGD and HGD from each other, suggesting that AMACR might be a useful diagnostic discriminator (23, 24). However, the sensitivity (less than 70%) does not allow a clinical use of AMACR. P53 was also analyzed because of its association with dysplasia (25, 26). However, p53 was never used in BE diagnosis because of the low specificity and sensitivity results observed in the IHC staining (27, 28). Few studies have been published previously using a panel of markers for BE progression or staging of the disease. Bird-Lieberman et al. identified a panel of seven biomarkers analyzed in a population based study that increase the accuracy of the prediction compared with any individual marker (29). All the markers discussed above are part of different pathways from those studied here and, if desired, could be explored further to complement the four markers of this disclosure.

The majority of the samples used in this study came from middle-aged men, which is concordant with the previous epidemiologic studies of BE (30). These samples were collected as part of the routine clinical care and hence are applicable to everyday practice and allow an adaptation of the treatment for each patient. Based on our findings, it may be beneficial to use these markers for diagnosis in BE disease as an adjunct, especially in diagnostically challenging cases to aid in the accurate diagnosis.

It will be recognized from this disclosure that CDX2, p120ctn, c-Myc and Jagged1 are characterized by a change in their expression between ND-BE/LGD and HGD/EAC. We showed that a combinatorial approach could successfully stratify patients into early and late stage categories and has the potential to optimize patient care.

### EXAMPLE 2

This Example provides a description of the materials and methods used to obtain that data described above. In the results described in this Example, samples analyzed were obtained from either: 1) pinch biopsies of the esophagus using forceps passed through the endoscope of patients undergoing an esophagogastroduodenoscopy (EGD), or 2) a surgical tissue sample excised from a patient during an esophagectomy procedure.

### Cases

This research was approved by the Hershey Medical Center Institutional Review Board. The surgical pathology files of the Hershey Medical Center were searched for ND-BE, dysplastic BE, and EAC cases diagnosed between 2001 and 2014 with available formalin-fixed, paraffin embedded tissue blocks. All the Hematoxylin and Eosin (H&E)-stained slides were reviewed a second time by a gastrointestinal (GI) pathologist for confirmation of the original diagnosis. Barrett's esophagus as defined per the American College of Gastroenterology necessitates both histologic (intestinal metaplasia with goblet cells) and endoscopic (columnar-type mucosa) features (4). Only samples with an agreement between the two anatomic pathologists (the pathologist who incurred the original diagnosis that diagnosed the patient and the second GI pathologist) were used for this study. Using those criteria, a total of 101 patients were selected for immunohistochemical staining. The tissues were classified as ND-BE, BE with LGD, BE with HGD and EAC (including intramucosal and submucosal esophageal adenocarcinoma).

### Immunohistochemistry

Sections of 5 μm were used for immunohistochemical analysis. For CDX2, p120ctn and c-Myc immunohistochemical staining, tissue sections were baked 1 hour at 55° C., deparaffinized with xylene and antigens were unmasked by heating in citrate buffer (0.01 M, pH 6.0). Endogenous peroxidase activity was blocked by incubation with 3% peroxide for 6 min. The slides were incubated overnight at 4° C. with primary antibodies (CDX2: Biogenex, Fremont, Calif., # Mu392A-UC, dilution 1:50; p120ctn: BD Biosciences, San Jose, Calif., # 610134, dilution 1:100; c-Myc: Epitomics Inc., Burlingame, Calif., # 1472-1, dilution 1:100). For antibody detection, the appropriate ImmPRESS anti-rabbit or anti-mouse reagent was used according to the manufacturer's protocol. Slides were incubated with DAB for 10 min and counterstained with Molecular hematoxylin prior to coverslipping with Permount. For Jagged1 staining, the IHC was performed by the Penn State COM Morphologic and Pathology Core Research Lab on an automated Discovery XT stainer, using an EDTA based retrieval solution (Ventana Medical System, Tucson, Ariz.). The slides were incubated overnight with Jagged1 antibody at a 1:400 dilution (Jagged1: Sigma Aldrich, Saint Louis, Mo., #HPA021555).

### Immunohistochemical Staining Evaluation

The sections were evaluated on an Olympus BX53 light microscope at 100×200× and 400× magnification and images were captured by an Olympus DP25 camera and the Olympus CellSens Dimension software. The staining intensity was graded semi-quantitatively at 200× with scores of 0 (absent), 1 (weak), 2 (moderate) or 3 (strong). The intensity was evaluated only if more than 10% of the cells were stained in the sample. An immunoreactivity score (IRS) was calculated (15, 18) as the staining intensity value (0 to 3) multiplied by the estimated value of the percentage of positively stained cells, determined as follows: 1 for 10% to 25% positive; 2 for 26% to 50% positive; 3 for 51% to 75% positive and 4, more than 75% positive. The total IRS ranged from 0 to 12. Thus, in an embodiment, the present disclosure includes calculating an IRS score from a sample. The calculation can be performed using the following equation, or any equation that provides an equivalent of the IRS:

SIV×PSC=IRS, wherein SIV is staining intensity value and PSC is percentage of positively stained cells. In embodiments, the disclosure includes performing the IRS calculation by use of a machine, such as a computer comprising a processor and software to produce the IRS. In embodiments, the IRS value is used categorized as either above or below a threshold value, which can be used for a treatment decision, or for diagnosis or aiding in the diagnosis, or staging of, for example, early stage (ND-BE/LGD) and late stage (HGD/EAC) disease.

### Statistical Analysis

The two groups early stage (ND-BE/LGD) and late stage (HGD/EAC) were analyzed using the Student's t-test. PCA was applied to the four immunohistochemical staining scores to reduce the dimension to two principal components. The two principal components were graphed and colored according to the clinical status of the patients (early stage versus late stage). The first principal component versus clinical status was separately graphed as a boxplot. To evaluate the utility of the potential marker, a ROC curve was constructed to assess the prediction ability to identify early from late stage patients using the scores of the immunostaining for the four proteins. All tests were carried out at a significance level of 0.05. The statistical analysis was performed using R version 3.0.0 (www.r-project.org)
1. Jankowski J A, Wright N A, Meltzer S J, et al. Molecular evolution of the metaplasia-dysplasia-adenocarcinoma sequence in the esophagus. Am J Pathol. 1999; 154(4):965-73. Epub 1999 May 11.
2. DeMeester S R. Evaluation and treatment of superficial esophageal cancer. J Gastrointest Surg. 2010; 14 Suppl 1:S94-100. Epub 2009 Sep. 18.
3. Bennett C, Vakil N, Bergman J, et al. Consensus Statements for Management of Barrett's Dysplasia and Early-Stage Esophageal Adenocarcinoma, Based on a Delphi Process. Gastroenterology. 2012; 143(2):336-46. Epub 2012 Apr. 28.
4. Wang K K, Sampliner R E. Updated guidelines 2008 for the diagnosis, surveillance and therapy of Barrett's esophagus. The American journal of gastroenterology. 2008; 103(3):788-97. Epub 2008 Mar. 18.
5. Montgomery E, Bronner M P, Goldblum J R, et al. Reproducibility of the diagnosis of dysplasia in Barrett esophagus: a reaffirmation. Human pathology. 2001; 32(4):368-78. Epub 2001 May 2.
6. Wani S, Falk G W, Post J, et al. Risk factors for progression of low-grade dysplasia in patients with Barrett's esophagus. Gastroenterology. 2011; 141(4):1179-86, 86 e1. Epub 2011 Jul. 5.
7. Kerkhof M, van Dekken H, Steyerberg E W, et al. Grading of dysplasia in Barrett's oesophagus: substantial interobserver variation between general and gastrointestinal pathologists. Histopathology. 2007; 50(7):920-7. Epub 2007 Jun. 5.
8. Denlinger C E, Thompson R K. Molecular basis of esophageal cancer development and progression. The Surgical clinics of North America. 2012; 92(5):1089-103. Epub 2012 Oct. 3.
9. Morales C P, Souza R F, Spechler S J. Hallmarks of cancer progression in Barrett's oesophagus. Lancet. 2002; 360(9345):1587-9. Epub 2002 Nov. 22.
10. Guo R J, Suh E R, Lynch J P. The role of Cdx proteins in intestinal development and cancer. Cancer biology & therapy. 2004; 3(7):593-601. Epub 2004 May 12.
11. Hayes S, Ahmed S, Clark P Immunohistochemical assessment for Cdx2 expression in the Barrett metaplasia-dysplasia-adenocarcinoma sequence. Journal of clinical pathology. 2011; 64(2):110-3. Epub 2010 Nov. 26.
12. Wijnhoven B P, Pignatelli M, Dinjens W N, Tilanus H W. Reduced p120ctn expression correlates with poor survival in patients with adenocarcinoma of the gastroesophageal junction. Journal of surgical oncology. 2005; 92(2):116-23. Epub 2005 Oct. 19.
13. Shaheen N J, Sharma P, Overholt B F, et al. Radiofrequency ablation in Barrett's esophagus with dysplasia. The New England journal of medicine. 2009; 360(22):2277-88. Epub 2009 May 29.
14. Stairs D B, Bayne L J, Rhoades B, et al. Deletion of p120-catenin results in a tumor microenvironment with inflammation and cancer that establishes it as a tumor suppressor gene. Cancer cell. 2011; 19(4):470-83. Epub 2011 Apr. 13.
15. Remmele W, Stegner H E. [Recommendation for uniform definition of an immunoreactive score (IRS) for immunohistochemical estrogen receptor detection (ER-ICA) in breast cancer tissue]. Der Pathologe. 1987; 8(3):138-40. Epub 1987 May 1. Vorschlag zur einheitlichen Definition eines Immunreaktiven Score (IRS) fur den immunhistochemischen Ostrogenrezeptor-Nachweis (ER-ICA) im Mammakarzinomgewebe.
16. Millikan K W, Silverstein J, Hart V, et al. A 15-year review of esophagectomy for carcinoma of the esophagus and cardia. Arch Surg. 1995; 130(6):617-24. Epub 1995 Jun. 1.
17. Shaheen N J. Advances in Barrett's esophagus and esophageal adenocarcinoma. Gastroenterology. 2005; 128(6):1554-66. Epub 2005 May 12.
18. Ditsch N, Toth B, Mayr D, et al. The association between vitamin D receptor expression and prolonged overall survival in breast cancer. The journal of histochemistry and cytochemistry: official journal of the Histochemistry Society. 2012; 60(2):121-9. Epub 2011 Nov. 24.
19. Pohl H, Welch H G. The role of overdiagnosis and reclassification in the marked increase of esophageal adenocarcinoma incidence. Journal of the National Cancer Institute. 2005; 97(2):142-6. Epub 2005 Jan. 20.
20. Revilla-Nuin B, Papilla P, Lozano J J, et al. Predictive value of MicroRNAs in the progression of barrett esophagus to adenocarcinoma in a long-term follow-up study. Ann Surg. 2013; 257(5):886-93. Epub 2012 Oct. 13.
21. Thrift A P, Kendall B J, Pandeya N, Vaughan T L, Whiteman D C. A clinical risk prediction model for Barrett esophagus. Cancer Prey Res (Phila). 2012; 5(9):1115-23. Epub 2012 Jul. 13.
22. van de Winkel A, van Zoest K P, van Dekken H, Moons L M, Kuipers E J, van der Laan L J. Differential expression of the nuclear receptors farnesoid X receptor (FXR) and pregnane X receptor (PXR) for grading dysplasia in patients with Barrett's oesophagus. Histopathology. 2011; 58(2):246-53. Epub 2011 Feb. 18.
23. Dorer R, Odze R D. AMACR immunostaining is useful in detecting dysplastic epithelium in Barrett's esophagus, ulcerative colitis, and Crohn's disease. The American journal of surgical pathology. 2006; 30(7):871-7. Epub 2006 Jul. 5.
24. Lisovsky M, Falkowski O, Bhuiya T. Expression of alpha-methylacyl-coenzyme A racemase in dysplastic Barrett's epithelium. Human pathology. 2006; 37(12):1601-6. Epub 2006 Sep. 26.
25. Jones D R, Davidson A G, Summers C L, Murray G F, Quinlan D C. Potential application of p53 as an intermediate biomarker in Barrett's esophagus. The Annals of thoracic surgery. 1994; 57(3):598-603. Epub 1994 Mar. 1.
26. Younes M, Lebovitz R M, Lechago L V, Lechago J. p53 protein accumulation in Barrett's metaplasia, dysplasia, and carcinoma: a follow-up study. Gastroenterology. 1993; 105(6):1637-42. Epub 1993 Dec. 1.
27. Coggi G, Bosari S, Roncalli M, et al. p53 protein accumulation and p53 gene mutation in esophageal carcinoma. A molecular and immunohistochemical study with clinicopathologic correlations. Cancer. 1997; 79(3):425-32. Epub 1997 Feb. 1.
28. Hamelin R, Flejou J F, Muzeau F, et al. TP53 gene mutations and p53 protein immunoreactivity in malignant and premalignant Barrett's esophagus. Gastroenterology. 1994; 107(4):1012-8. Epub 1994 Oct. 1.
29. Bird-Lieberman E L, Dunn J M, Coleman H G, et al. Population-based study reveals new risk-stratification biomarker panel for Barrett's esophagus. Gastroenterology. 2012; 143(4):927-35 e3. Epub 2012 Jul. 10.
30. Ronkainen J, Aro P, Storskrubb T, et al. Prevalence of Barrett's esophagus in the general population: an endoscopic study. Gastroenterology. 2005; 129(6):1825-31. Epub 2005 Dec. 14.

While the invention has been described through specific embodiments, routine modifications will be apparent to those skilled in the art and such modifications are intended to be within the scope of the present invention.

What is claimed is:

1. A method for staging an esophageal condition in an individual at risk for or suspected of having the esophageal condition comprising testing a biological sample from the individual for expression of CDX2, p120ctn, c-Myc and Jagged1 proteins, comparing the amount of the CDX2, p120ctn, c-Myc and Jagged1 proteins to reference values, and providing a diagnosis of, or aiding in a physician's diagnosis of, the individual as having high-grade dysplasia (HGD) or esophageal adenocarcinoma (EAC) by determining:
i) less CDX2 protein relative to non-dysplastic Barrett's esophagus (ND-BE) and low-grade dysplasia (LGD) CDX2 protein values, but more CDX2 protein than a normal CDX2 protein reference value; and
ii) less p120ctn protein relative to ND-BE, LGD and normal 120ctn protein reference values; and
iii) increased c-Myc protein relative to ND-BE and LGD protein reference values; and
iv) increased Jagged1 protein relative to normal and ND-BE Jagged1 protein reference values.

2. The method of claim 1, wherein the CDX2 protein, the p120ctn protein, the c-Myc protein, and the Jagged1 protein are determined using immunohistochemistry.

3. The method of claim 1, wherein the normal CDX2 protein value, the normal 120ctn protein reference value, and the normal Jagged1 protein reference value are determined from one or more individuals who do not have ND-BE, LGD, HGD or EAC.

4. The method of claim 1, wherein the biological sample is a sample of esophageal tissue.

5. The method of claim 1, further comprising performing an ablative technique on, or surgical resection of, a portion of the esophagus of the individual, wherein the portion of the esophagus comprises cells that exhibit i), ii), iii) and iv) of claim 1.

6. The method of claim 5, wherein the ablative technique is performed.

7. The method of claim 1, further comprising fixing the determination of the CDX2 protein, the p120ctn protein, the c-Myc protein and the Jagged1 protein in a tangible medium.

8. The method of claim 7, wherein the determination is expressed as an immunoreactivity score intensity.

9. The method of claim 7, wherein the tangible medium is provided to a health care provider.

10. A kit for use in diagnosis of high-grade dysplasia (HGD) and/or esophageal adenocarcinoma (EAC) comprising a) a monoclonal antibody that is specific for CDX2 protein; b) a monoclonal antibody that is specific for p120ctn protein, c) a monoclonal antibody that is specific for c-Myc protein; and a d) monoclonal antibody that is specific for Jagged1 protein.

11. The kit of claim 10, wherein the monoclonal antibodies of a), b), c) and d) are primary antibodies for use with secondary detection antibodies, wherein the secondary detection antibodies are detectably labeled.

12. The kit of claim 10, wherein the antibodies of a), b), c) and d) are the only monoclonal antibodies in the kit.

13. The kit of claim 11, further comprising the secondary antibodies, wherein the detectable label comprises an enzyme.

14. The kit of claim 13, wherein the kit further comprises one or more reagents for detecting the secondary antibodies.

15. The kit of claim 14, wherein the one or more reagents comprises a substrate that can be modified by the enzyme to produce a detectable signal.
